(19) United States
(12) Patent Application Publication
(10) Pub. No.: US 2009/0008447 A1
(43) Pub. Date: Jan. 8, 2009

(54) METHOD AND SYSTEM FOR MANAGING INVENTORY IN A HEALTHCARE FACILITY

(76) Inventor: Peter Phillip Godlewski, San Clemente, CA (US)

Correspondence Address: MCDERMOTT, WILL & EMERY, 4370 LA JOLLA VILLAGE DRIVE, SUITE 700, SAN DIEGO, CA, 92122, US

(21) Appl. No.: 12/135,091

(22) Filed: Jun. 6, 2008

Related U.S. Application Data
(60) Provisional application No. 60/942,593, filed on Jun. 7, 2007.

(51) Int. Cl. G06Q 50/00

(52) U.S. Cl. 235/385

(57) ABSTRACT

A method and system for managing inventory in a healthcare facility, in which it is determined whether a machine-readable tag affixed to at least one of a used item and a container of the used item is deposited in a receptacle. It is automatically communicated to a centralized database that the machine-readable tag has been deposited in the receptacle. A demand signal may be automatically generated demanding a replacement for the used item, when it is determined that the machine-readable tag is deposited in the receptacle. Also, a charge for the used item may be automatically generated on a patient bill stored in the database, when it is determined that the machine-readable tag is deposited in the receptacle.

### CLAIM OF PRIORITY UNDER 35 USC § 119

The present application claims priority to U.S. Provisional Application No. 60/942,593, entitled “Method and System for Smart Order Replenishment and Charge Capture”, filed Jun. 7, 2007, having attorney docket number CTS094, and which is incorporated herein by reference in its entirety.

### FIELD

The present disclosure relates generally to systems and method for managing inventory in a healthcare facility.

### BACKGROUND

When a patient is treated in a healthcare facility, various items, such as instruments and medication, may be used during the course of treatment. A nurse or other healthcare provider, such as a doctor or pharmacist, needs to capture billing information for procedures and items used during the treatment. Conventionally, however, options for capturing charges include barcode scanning or manually inputting charges via a keyboard or other device. In an operating room scenario, for example, the nurse may not have time or the means to capture the necessary charges. The conventional charge capturing mechanisms often result in inefficiencies and inaccurate billing to the patient.

In addition, a supplier of an item used during treatment of a patient faces stiff competition with other suppliers, and it may be difficult to maintain long-term accounts with a healthcare facility. Based on conventional methods of managing inventory, there are virtually no barriers for healthcare facilities to erratically convert to another supplier of a particularly item.

Therefore, there is a need in the art for a method and system for improved management of inventory in a healthcare facility.

### SUMMARY

The presently disclosed embodiments are directed to solving one or more of the problems presented in the prior art, described above, as well as providing additional features that will become readily apparent by reference to the following detailed description when taken in conjunction with the accompanying drawings.

One or more aspects of the present disclosure are directed to a method of managing inventory in a healthcare facility. The method comprises determining whether a machine-readable tag affixed to at least one of a used item and a container of the used item is deposited in a receptacle; and automatically communicating to a centralized database that the machine-readable tag has been deposited in the receptacle.

One or more other aspects of the present disclosure are directed to a system of managing inventory in a healthcare facility. The system comprises a receptacle configured to determine whether a machine-readable tag affixed to at least one of a used item and a container of the used item is deposited in a receptacle, and to automatically communicating to a centralized database that the machine-readable tag has been deposited in the receptacle.

One or more aspects of the present disclosure are directed to a computer-readable medium storing instructions thereon for performing a method of managing inventory in a healthcare facility. The method comprises determining whether a machine-readable tag affixed to at least one of a used item and a container of the used item is deposited in a receptacle; and automatically communicating to a centralized database that the machine-readable tag has been deposited in the receptacle.

As will become evident by the following Description and Drawings, determining whether a machine-readable tag affixed to at least one of a used item and a container of the used item is deposited in a receptacle, and automatically communicating to a centralized database that the machine-readable tag has been deposited in the receptacle, allows a supplier and/or a distributor of the used item to establish an ongoing relationship with customers based on convenience. That is, the supplier or distributor can ensure that the healthcare facility will continue to stock the used item from them, even in a competitive market, since the burden associated with replacing hardware in the healthcare facility and/or retraining healthcare facility staff to implement another item-replacement process will be far outweighed by the convenience of automatically reordering the used item from the current supplier or distributor. Moreover, a patient bill may be automatically generated to include a charge for the used item, since the centralized database knows what item was used, based on the machine-readable tag deposited in the receptacle.

Of course, the present invention is not limited to the aforementioned embodiments, and other features of the embodiments will become apparent after review of the hereinafter set forth Brief Description of the Drawings, Detailed Description, and the Claims, or may be learned by practice of the invention.

### BRIEF DESCRIPTION OF THE DRAWINGS

The foregoing aspects of the embodiments described herein will become more readily apparent by reference to the following detailed description when taken in conjunction with the accompanying drawings wherein:

FIG. 1 is a graphical illustration of a system for managing inventory in a healthcare facility, according to one or more disclosed embodiments.

FIG. 2 is a graphical illustration of a container of an item with a machine-readable tag affixed thereto, according to one or more disclosed embodiments.

FIG. 3 is a flow diagram showing a method for managing inventory in a healthcare facility, according to one or more disclosed embodiments.

FIG. 4 is a flow diagram showing a method for determining whether a machine-readable tag has been deposited in a receptacle and for associating the machine-readable tag with a used item, according to one or more disclosed embodiments.

FIG. 5 is a flow diagram showing a method for automatically generating a demand signal demanding a replacement for a used item, according to one or more disclosed embodiments.

FIG. 6 is a flow diagram showing a method for automatically generating a charge for a used item on a patient bill, according to one or more disclosed embodiments.

### DETAILED DESCRIPTION

Reference will now be made in detail to the presently disclosed embodiments, examples of which are illustrated in the accompanying drawings, wherein like reference numerals refer to like elements throughout.

In the following detailed description, numerous specific details are set forth to provide a full understanding of the subject technology. It will be obvious, however, to one ordinarily skilled in the art that the subject technology may be practiced without some of these specific details. In other instances, well-known structures and techniques have not been shown in detail so as not to obscure the subject technology.

Embodiments of the present disclosure address and solve problems related to inventory management, including that of capturing billing information for used items in a healthcare facility. In addition, suppliers have difficulty keeping accounts, since there are no barriers to conversion from one supplier to a competing one. These and other concerns are solved, at least in part, by embodiments of the present invention that provide, for example, a method and system for inventory management in a healthcare facility. The method and system include determining whether at least one of a used item and a container of the used item is deposited in a receptacle, based on a machine-readable tag affixed to the at least one of the used item and the container of the used item, and communicating to a centralized database that at least one of the used item and the container of the used item has been deposited in the receptacle. As a result, a supplier of the item can ensure that the healthcare facility will continue to stock items from the supplier in a competitive market. The healthcare facility, for its part, assures adequate and timely restocking of supplies in an automatic manner, reducing inventory checks. Moreover, a patient bill may be automatically generated to include a charge for the used item, since the centralized database knows what item was used, based on the machine-readable tag deposited in the receptacle.

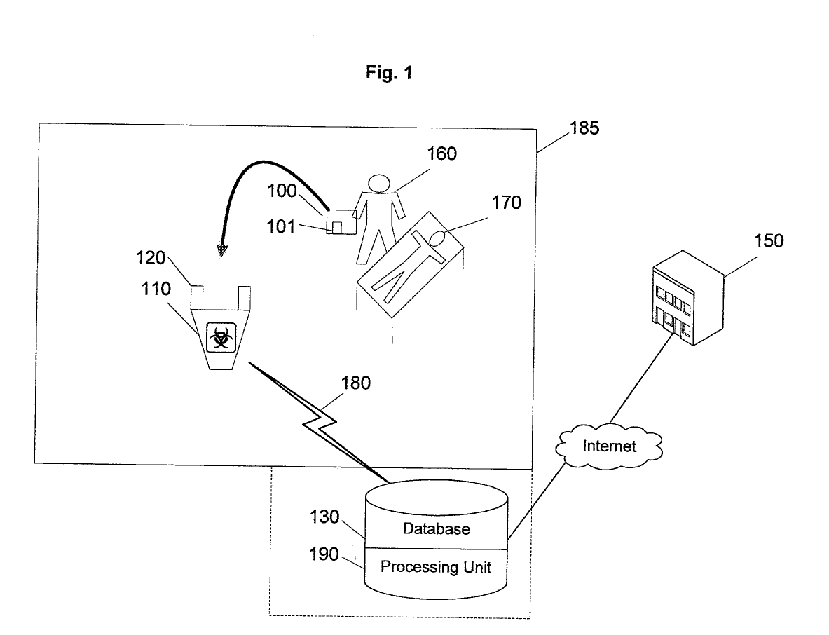

FIG. 1 is a graphical illustration of a system for managing inventory in a healthcare facility, according to one or more disclosed embodiments. In an operating room, for example, a healthcare facility staff member (e.g., a nurse) 160 may activate a database 130 (e.g., a Pyxis® station) so that the database 130 knows that the operating room is being used for a particular patient 170. The database 130 may be a centralized database within the healthcare facility 185, for example, or may be located remote from the facility. For illustrative purposes, the dotted line, indicates that the database 130 may be within healthcare facility 185 or at some other location. Alternatively, the reporting may be to a local database, which then provides its stored information to a centralized database. When the staff member 160 uses an item 100 from a casepack, for example, in treating a patient 170, the used item 100 or the container (e.g., wrapper) of the used item 100 is deposed into a receptacle 110, such as a trash can or a biohazard receptacle 110. Upon entering the receptacle 110, a sensor 120 senses a machine-readable tag 101 that was previously affixed to the used item 100. According to certain embodiments, the machine-readable tag 101 is previously affixed to the used item 100 by a supplier 150 before being supplied to the healthcare facility, although in other embodiments, the machine-readable tag 101 is affixed after the item reaches the healthcare facility. It is noted that the term “machine-readable tag” is used throughout the present disclosure for exemplary purposes. However, the machine-readable tag 101 may be any readable label bearing a unique identification that is associated with the used item 100. For example, the machine-readable tag may be a 3D barcode, a radio frequency identification (RFID) tag, a magnetically encoded tag, a chip etc. Accordingly, sensor 120 may be an RFID antenna, for example, or any sensing device capable of detecting the machine-readable tag 101.

The sensor 120 transmits a signal to the database 130, via a connection 180, which indicates that the machine readable tag 101 (affixed to the used item 100, for example) has been deposited in the receptacle 110. The connection 180 may be a wireless connection (e.g., WiFi) or a hard-wired connection (e.g., a local area network). According to certain embodiments, the sensor 120 is incorporated into a local area network and/or wireless local area network, complying with IEEE 802.11 standards. According to other embodiments, the sensor 120 may communicate with the database 130 via the internet. Any conventional means of communication, however, may be employed between the sensor 120 and the database 130. For example, although a direct connection to the database 130 is illustrated, it will be understood by those of skill in the art that various processing devices (not shown) serve to provide the connectivity required, and to manage the database 130.

The signal transmitted from the sensor 120 to the database 130 may provide, for example, a unique identifier of the machine readable tag 101. A processing unit 190 communicatively coupled to the database 130 associates the machine readable tag 101 with the used item 100. According to an embodiment, the processing unit 190 communicatively coupled to the database 130 may automatically generate a charge for the used item on a patient bill stored in the database 130. Thus, the healthcare facility staff member 160 is not required to capture charges by scanning a barcode or typing any information into a device using a keyboard, for example.

According to certain embodiments, the database 130 may automatically generate a demand signal to send to a supplier 150 of the used item 100, when it is determined that the machine-readable tag 101 affixed to the used item 100 has been deposited in the receptacle 110 based on the signal transmitted from the sensor 120. The demand signal may be sent, for example, via the internet or any conventional means of communication. The demand signal requests a replacement for the used item 100 from the supplier 150. According to certain embodiments, the supplier 150 may maintain a database (not shown) of which items 100 (e.g., casepacks) are associated with each machine-readable tag 101 sensed by the sensor 120. Therefore, the signal transmitted from the database 130 to the supplier 150 only needs to identify the machine-readable tag 101 sensed by the sensor 120. Thus, the supplier 150 of the used item 100 is more likely to keep a constant account with a healthcare facility, since the healthcare facility 185 is less likely to convert from the current supplier 150 to a competitor, for example.

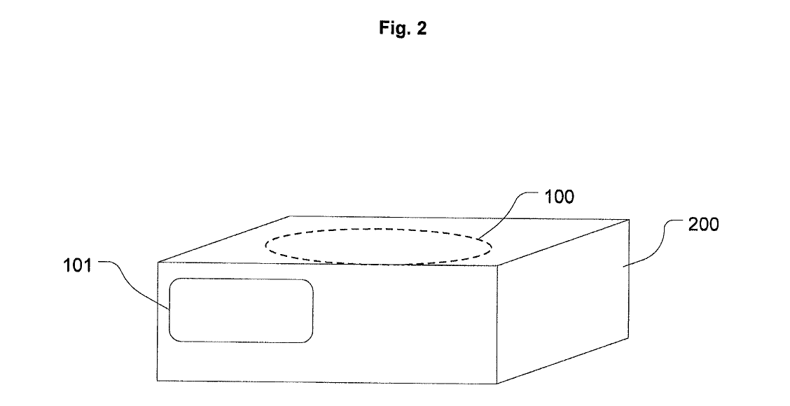

FIG. 2 is a graphical illustration of a container 200 of an item 100 with a machine-readable tag 101 affixed thereto, according to one or more disclosed embodiments. According to certain aspects of the present disclosure, the used item 100 itself may not be disposed of in receptacle 110. In these cases, a container 200 of the used item 100 includes the machine-readable tag 101 affixed thereto. When the container 200 is deposited in the receptacle 110, the sensor 120 senses the affixed machine-readable tag 101 and transmits a signal to the database 130, as described above with reference to FIG. 1. The container 200 may be any storage device or wrapper, for example, associated with the used item 100. The machine-readable tag 101 need not be actually affixed to the surface of the container 200, but may be contained within the container 200.

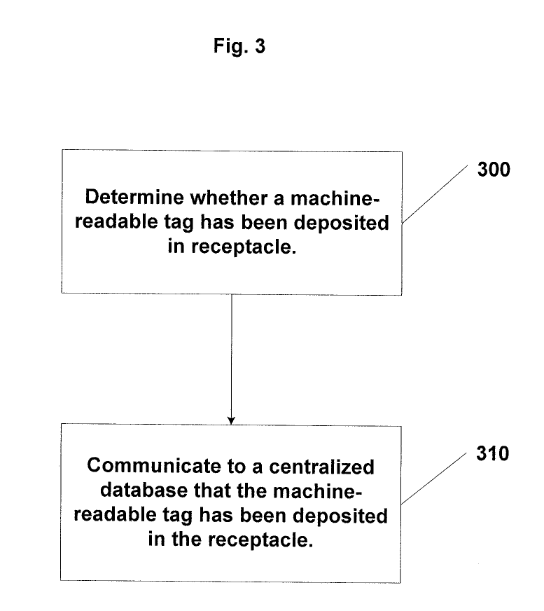

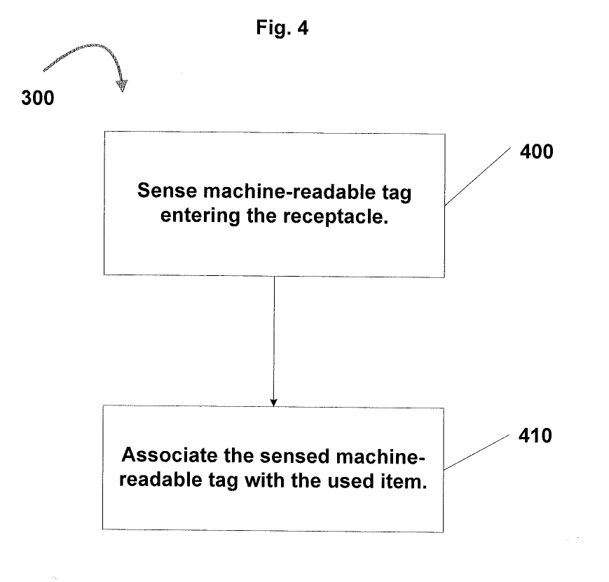

FIG. 3 is a flow diagram showing a method for managing inventory in a healthcare facility, according to one or more disclosed embodiments. At operation 300, it is determined whether a machine-readable tag 101 has been deposited in the receptacle 110. The process of determining whether the machine-readable tag 101 has been deposited in the receptacle 110, according to various embodiments, is described in further detail with reference to operation 400 in FIG. 4 below.

From operation 300, the process continues to operation 310, where it is communicated to the database 130 that the machine-readable tag 101 has been deposited in the receptacle 110. As described above, the sensor 120 transmits a signal to the database 130, via a connection 180, which indicates that the machine readable tag 101, which is affixed to the used item 100 or the container 200 of the used item 100, for example, has been deposited in the receptacle 110.

FIG. 4 is a flow diagram showing a method for determining whether a machine-readable tag 101 has been deposited in a receptacle 110 and for associating the machine-readable tag 101 with a used item 100, according to one or more disclosed embodiments. At operation 400, a sensor 120 senses that the machine-readable tag 101 enters the receptacle 110. As described above, the sensor 120 may be an RFID antenna, which senses when an RFID tag, for example, is in close proximity. It is noted that the machine-readable tag 101 is not limited to an RFID tag, but may be any readable label bearing a unique identification that is associated with the used item 100.

Once the sensor 120 senses that the machine-readable tag 101 has entered the receptacle 110, it is communicated to the database 130 that the machine-readable tag 101 has been deposited in the receptacle 110, as described above with reference to operation 310 of FIG. 3. As also described above, sensor 120 communicates with database 130 via the connection 180, which may be, for example, a wireless connection (e.g., WiFi) or a hard-wired connection (e.g., a local area network). However, any conventional means of communication, may be employed between the sensor 120 and the database 130.

According to certain embodiments, after it is communicated from the sensor 120 to the database 130 that the machine-readable tag 101 has been deposited in the receptacle (at operation 310), the process continues to operation 410 where a processing unit 190 communicatively coupled to database 130 associates the sensed machine-readable tag 101 with the used item 100. Various other information regarding the sensed machine-readable tag 101 may be pre-programmed into the database 130. This information may include, for example, a chargeable amount of the used item 100, supplier 150 information, etc.

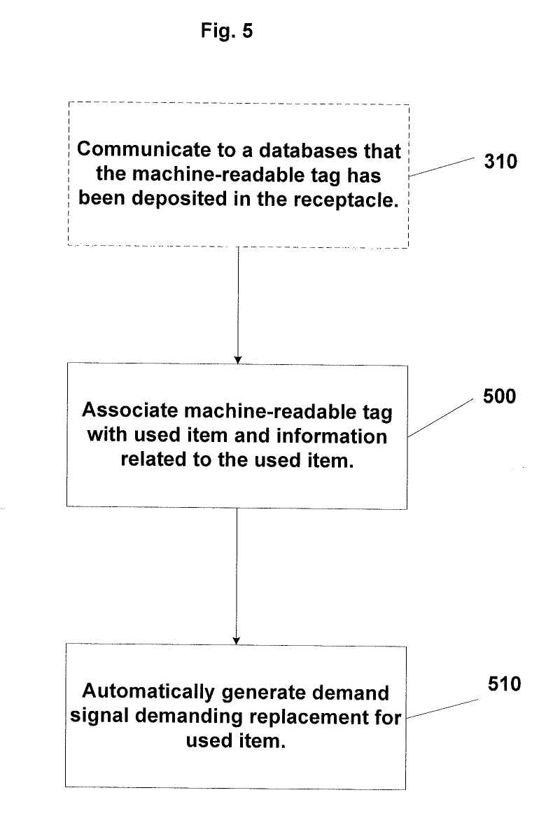

FIG. 5 is a flow diagram showing a method for automatically generating a demand signal demanding a replacement for a used item 100, according to one or more disclosed embodiments. After it is communicated from the sensor 120 to the centralized database 130 that a machine-readable tag 101 has been deposited in the receptacle 110, at operation 500, a processing unit 190 communicatively coupled to the database 130 associates the sensed machine-readable tag 101 with the used item 100. Various other information regarding the sensed machine-readable tag 101 may be pre-programmed into the database 130. This information may include, for example, a chargeable amount of the used item 100, supplier 150 information, etc.

From operation 500, the process may continue to operation 510 where the processing unit 190 automatically generates a demand signal demanding a replacement for the used item 100 from the supplier 150. According to an embodiment, the processing unit 190 may associate the used item 100 with a particular supplier 150 pre-programmed in the database 130. The demand signal may be sent, for example, via the internet or any conventional means of communication. According to certain embodiments, the supplier 150 may maintain a database (not shown) of which items 100 (e.g., casepacks) are associated with each machine-readable tag 101 sensed by the sensor 120. Therefore, the signal transmitted from the database 130 to the supplier 150 only needs to identify the machine-readable tag 101 sensed by the sensor 120.

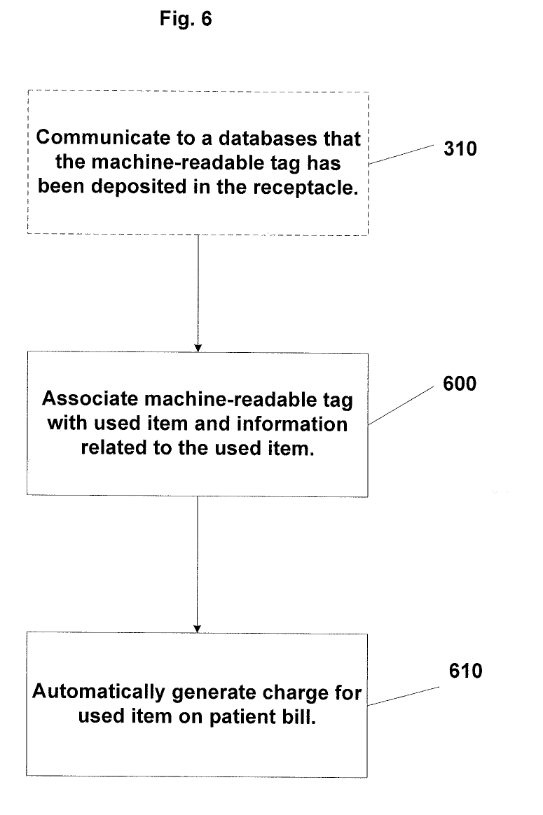

FIG. 6 is a flow diagram showing a method for automatically generating a charge for a used item 100 on a patient bill, according to one or more disclosed embodiments. After it is communicated from the sense 120 to the database 130 that a machine-readable tag 101 has been deposited in the receptacle 110, a processing unit 190 communicatively coupled to the centralized database 130, at operation 600, associates the sensed machine-readable tag 101 with the used item 100. Using the information pre-programmed into database 130, the processing unit 190 may automatically generate a charge on a patient bill stored in database 130. The chargeable amount of the used item 100 may be pre-programmed into database 130, such that once the appropriate used item 100 has been associated with the machine-readable tag 101 deposited in the receptacle 110, a charge of the used item 100 may also be assigned to the machine-readable tag 101 deposited in the receptacle 110. As noted above, the operating room, for example, in which the patient 170 is located is previously activated by a healthcare facility staff member (e.g., a nurse) 160 so that the database 130 knows that the operating room is being used for the particular patient 170. Accordingly, the charge may be accurately generated on the appropriate patient bill.

By determining whether a machine-readable tag 101, affixed to at least one of a used item 100 and a container 200 of the used item 100 is deposited in a receptacle 110, and automatically communicating to a centralized database 130 that the machine-readable tag 101 has been deposited in the receptacle 110, a supplier 150 of the used item 100 can ensure that the healthcare facility 185 will continue to stock the used item 100 from the supplier 150, even in a competitive market. Moreover, a patient bill may be automatically generated to include a charge for the used item 100, since the centralized database 130, for example, knows what item 100 was used, based on the machine-readable tag 101 deposited in the receptacle 110.

The methods and systems described herein solve challenges that suppliers and distributors face when healthcare facilities seek to convert to different suppliers or distributors in order to negotiate lower prices for various items, which causes suppliers to lose business and decreases returns on investments. By affixing the machine-readable tag 101 to the item 100 and supplying the receptacle 110 with the sensor 120, for example, a supplier 150 creates a stable, ongoing client relationship where the healthcare facility 185 is likely to reorder the item 100 from the same supplier 150.

Moreover, by automatically generating a charge on a patient bill, the healthcare facility 185 may easily capture charges. A healthcare facility staff member (e.g., a nurse) may not have time to use traditional methods of capturing charges, such as scanning barcodes or entering the charge via a keyboard. In addition, automatically generating the charge for the used item 100 avoids inaccurate billing resulting from traditional billing mechanisms.

The previous description is provided to enable any person skilled in the art to practice the various aspects described herein. Various modifications to these aspects will be readily apparent to those skilled in the art, and the generic principles defined herein may be applied to other aspects. Thus, the claims are not intended to be limited to the aspects shown herein, but is to be accorded the full scope consistent with the language claims, wherein reference to an element in the singular is not intended to mean “one and only one” unless specifically so stated, but rather “one or more.” Unless specifically stated otherwise, the term “some” refers to one or more.

Those of ordinary skill would further appreciate that the various illustrative logical modules, circuits and algorithms described in connection with the embodiments disclosed herein may be implemented as electronic hardware, computer software or combinations of both. To clearly illustrate this interchangeability of hardware and software, various illustrative components, blocks, modules, circuits and steps have been described above generally in terms of their functionality. Whether such functionality is implemented as hardware or software depends upon the particular application and design constraints imposed on the overall system. Skilled artisans may implement the described functionality in varying ways for each particular application, but such implementation decisions should not be interpreted as causing a departure from the scope of the present disclosure.

The various illustrative logical blocks, modules, and circuits described in connection with the embodiments disclosed herein may be implemented or performed with a general purpose processor, a digital signal processor (DSP), an application specific integrated circuit (ASIC), a filed programmable gate array (FPGA) or other programmable logic device, discrete gate or transistor logic, discrete hardware components, or any combination thereof designed to perform the functions described herein. A general purpose processor may be a microprocessor, but in the alternative, the processor may be any conventional process, control, microcontroller, or state machine. A process may also be implemented as a combination of computing devices, e.g., a combination of a DSP and a microprocessor, a plurality of microprocessors, one or more microprocessors in conjunction with a DSP core, or any other such configuration.

In one or more exemplary embodiments, the functions described may be implemented in hardware, software, firmware, or any combination thereof. If implemented in software, the functions may be stored on or transmitted over as one or more instructions or code on a computer-readable medium. Computer-readable media includes both computer storage media and communication media including any medium that facilitates transfer of a computer program from one place to another. A storage media may be any available media that can be accessed by a computer. By way of example, and not limitation, such computer-readable media can comprise RAM, ROM, EEPROM, CD-ROM or other optical disk storage, magnetic disk storage or other magnetic storage devices, or any other medium that can be used to carry or store desired program code in the form of instructions or data structures and that can be accessed by a computer. Also, any connection is properly termed a computer-readable medium. For example, if the software is transmitted from a website, server, or other remote source using a coaxial cable, fiber optic cable, twisted pair, digital subscriber line (DSL), or wireless technologies such as infrared, radio, and microwave, then the coaxial cable, fiber optic cable, twisted pair, DSL, or wireless technologies such as infrared, radio, and microwave are included in the definition of medium. Disk and disc, as used herein, includes compact disc (CD), laser disc, optical disc, digital versatile disc (DVD), floppy disk and blu-ray disc where disks usually reproduce data magnetically, while discs reproduce data optically with lasers. Combinations of the above should also be included within the scope of computer-readable media.

All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S.C. §112, sixth paragraph, unless the element is expressly recited using the phrase “means for” or, in the case of a method claim, the element is recited using the phrase “step for.”

It is understood that the specific order or hierarchy of steps or operations in the processes disclosed is an illustration of exemplary approaches. Based upon design preferences, it is understood that the specific order or hierarchy of steps or operations in the processes may be rearranged. Some of the steps may be performed simultaneously. The accompanying method claims present elements of the various steps or operations in a sample order, and are not meant to be limited to the specific order or hierarchy presented.

What is claimed is:

1. A method of managing inventory in a healthcare facility, comprising:
determining whether a machine-readable tag affixed to at least one of a used item and a container of the used item is deposited in a receptacle at the healthcare facility; and
automatically communicating to a centralized database that the machine-readable tag has been deposited in the receptacle.

2. The method of claim 1, further comprising sensing at the receptacle that the machine-readable tag has entered the receptacle.

3. The method of claim 2, further comprising associating at the centralized database the sensed machine-readable tag with the used item.

4. The method of claim 1, further comprising automatically generating a demand signal demanding a replacement for the used item, when it is determined that the machine-readable tag is deposited in the receptacle.

5. The method of claim 2, wherein the demand signal is generated at the centralized database communicatively coupled to the receptacle.

6. The method of claim 5, further comprising transmitting the demand signal from the centralized database to a supplier of the used item.

7. The method of claim 1, further comprising automatically generating a charge for the used item on a patient bill stored in the centralized database, when it is determined that the machine-readable tag is deposited in the receptacle.

8. The method of claim 1, wherein a supplier of the used item previously affixes the machine-readable tag to the at least one of the used item and the container of the used item.

9. The method of claim 1, wherein the machine-readable tag is a radio frequency identification (RFID) tag.

10. A system of managing inventory in a healthcare facility, comprising:
a receptacle configured to determine whether a machine-readable tag affixed to at least one of a used item and a container of the used item is deposited in the receptacle at the healthcare facility; and
a communication interface to automatically communicate to a centralized database that the machine-readable tag has been deposited in the receptacle.

11. The system of claim 10, wherein the communication interface comprises a sensor, at the receptacle, configured to sense that the machine-readable tag has entered the receptacle.

12. The system of claim 11, further comprising:
a processor at the centralized database associating the sensed machine-readable tag with the used item.

13. The system of claim 10, further comprising a processing unit configured to generate a demand signal demanding a replacement for the used item, when it is determined that the machine-readable tag is deposited in the receptacle.

14. The system of claim 13, wherein the demand signal is generated at the centralized database communicatively coupled to the receptacle.

15. The system of claim 14, further comprising a transmitter configured to transmit the demand signal from the centralized database to a supplier of the used item.

16. The system of claim 10, further comprising a processing unit configured to automatically generate a charge for the used item on a patient bill stored in the database, when it is determined that the machine-readable tag is deposited in the receptacle.

17. The system of claim 10, wherein a supplier of the used item previously affixes the machine-readable tag to the at least one of the used item and the container of the used item.

18. The system of claim 10, wherein the machine-readable tag is a radio frequency identification (RFID) tag.

19. A computer-readable medium storing instructions thereon for performing a method of managing inventory in a healthcare facility, the method comprising:
determining whether a machine-readable tag affixed to at least one of a used item and a container of the used item is deposited in a receptacle at the healthcare facility; and
automatically communicating to a centralized database that the machine-readable tag has been deposited in the receptacle.
